SEQUENCE LISTING (40 sequences)

SEQ ID NO: 1 - protein, 9 aa, synthetic construct
  MOD_RES 2: note = x is beta-Alanine
  MOD_RES 4: note = x is aminohexanoic acid
  MOD_RES 6: note = x is beta-Alanine
  MOD_RES 8: note = x is aminohexanoic acid
  MOD_RES 9: note = x is beta-Alanine
  source 1..9: note = peptide
RXRXRXRXX

SEQ ID NO: 2 - protein, 13 aa, synthetic construct
  MOD_RES 2: note = X is beta-Alanine
  MOD_RES 4: note = X is aminohexanoic acid
  MOD_RES 6: note = X is beta-Alanine
  MOD_RES 8: note = X is aminohexanoic acid
  MOD_RES 9: note = X is beta-Alanine
  MOD_RES 11: note = X is aminohexanoic acid
  MOD_RES 13: note = X is beta-Alanine
  source 1..13: note = peptide
RXRXRXRXXRXRX

SEQ ID NO: 3 - protein, 10 aa, synthetic construct
  MOD_RES 2: note = X is beta-Alanine
  MOD_RES 4: note = X is aminohexanoic acid
  MOD_RES 6: note = X is beta-Alanine
  MOD_RES 8: note = X is aminohexanoic acid
  MOD_RES 9: note = X is beta-Alanine
  source 1..10: note = peptide
RXRXRXRXXR

SEQ ID NO: 4 - protein, 14 aa, synthetic construct
  MOD_RES 2: note = X is beta-Alanine
  MOD_RES 5: note = X is aminohexanoic acid
  MOD_RES 7: note = X is beta-Alanine
  MOD_RES 9: note = X is aminohexanoic acid
  MOD_RES 10: note = X is beta-Alanine
  MOD_RES 12: note = X is aminohexanoic acid
  MOD_RES 14: note = X is beta-Alanine
  source 1..14: note = peptide
RXRRXRXRXXRXRX

SEQ ID NO: 5 - protein, 12 aa, synthetic construct
  MOD_RES 2: note = X is beta-Alanine
  MOD_RES 4: note = X is aminohexanoic acid
  MOD_RES 6: note = X is beta-Alanine
  MOD_RES 8: note = X is beta-Alanine
  MOD_RES 10: note = X is aminohexanoic acid
  MOD_RES 12: note = X is beta-Alanine
  source 1..12: note = peptide
RXRXRXRXRXRX

SEQ ID NO: 6 - protein, 12 aa, synthetic construct
  MOD_RES 2: note = X is beta-Alanine
  MOD_RES 4: note = X is aminohexanoic acid
  MOD_RES 6: note = X is beta-Alanine
  MOD_RES 8: note = X is beta-Alanine
  MOD_RES 10: note = X is beta-Alanine
  MOD_RES 12: note = X is beta-Alanine
  source 1..12: note = peptide
RXRXRXRXRXRX

SEQ ID NO: 7 - protein, 12 aa, synthetic construct
  MOD_RES 2: note = X is beta-Alanine
  MOD_RES 4: note = X is beta-Alanine
  MOD_RES 6: note = X is beta-Alanine
  MOD_RES 8: note = X is beta-Alanine
  MOD_RES 10: note = X is beta-Alanine
  MOD_RES 12: note = X is beta-Alanine
  source 1..12: note = peptide
RXRXRXRXRXRX

SEQ ID NO: 8 - protein, 11 aa, synthetic construct
  MOD_RES 2: note = X is beta-Alanine
  MOD_RES 4: note = X is aminohexanoic acid
  MOD_RES 6: note = X is beta-Alanine
  MOD_RES 8: note = X is beta-Alanine
  MOD_RES 10: note = X is aminohexanoic acid
  source 1..11: note = peptide
RXRXRXRXRXR

SEQ ID NO: 9 - protein, 12 aa, synthetic construct
  MOD_RES 2: note = x is beta-Alanine
  MOD_RES 5: note = x is beta-Alanine
  MOD_RES 7: note = x is beta-Alanine
  MOD_RES 9: note = x is beta-Alanine
  MOD_RES 12: note = x is beta-Alanine
  source 1..12: note = peptide
RXRRXRXRXRRX

SEQ ID NO: 10 - protein, 14 aa, synthetic construct
  MOD_RES 2: note = X is beta-Alanine
  MOD_RES 5: note = X is beta-Alanine
  MOD_RES 7: note = X is beta-Alanine
  MOD_RES 9: note = X is aminohexanoic acid
  MOD_RES 10: note = X is beta-Alanine
  MOD_RES 12: note = X is aminohexanoic acid
  MOD_RES 14: note = X is beta-Alanine
  source 1..14: note = peptide
RXRRXRXRXXRXRX

SEQ ID NO: 11 - protein, 14 aa, synthetic construct
  MOD_RES 2: note = X is beta-Alanine
  MOD_RES 5: note = X is beta-Alanine
  MOD_RES 7: note = X is beta-Alanine
  MOD_RES 9: note = X is beta-Alanine
  MOD_RES 10: note = X is beta-Alanine
  MOD_RES 12: note = X is aminohexanoic acid
  MOD_RES 14: note = X is beta-Alanine
  source 1..14: note = peptide
RXRRXRXRXXRXRX

SEQ ID NO: 12 - protein, 14 aa, synthetic construct
  MOD_RES 2: note = X is beta-Alanine
  MOD_RES 5: note = X is beta-Alanine
  MOD_RES 7: note = X is beta-Alanine
  MOD_RES 9: note = X is beta-Alanine
  MOD_RES 10: note = X is beta-Alanine
  MOD_RES 12: note = X is beta-Alanine
  MOD_RES 14: note = X is beta-Alanine
  source 1..14: note = peptide
RXRRXRXRXXRXRX

SEQ ID NO: 13 - protein, 5 aa, synthetic construct
  source 1..5: note = peptide
YQFLI

SEQ ID NO: 14 - protein, 5 aa, synthetic construct
  source 1..5: note = peptide
FQILY

SEQ ID NO: 15 - protein, 5 aa, synthetic construct
  source 1..5: note = peptide
ILFQY

SEQ ID NO: 16 - protein, 19 aa, synthetic construct
  MOD_RES 7: note = X is beta-Alanine
  MOD_RES 9: note = X is aminohexanoic acid
  MOD_RES 11: note = X is beta-Alanine
  MOD_RES 13: note = X is aminohexanoic acid
  MOD_RES 14: note = X is beta-Alanine
  source 1..19: note = peptide
YQFLIRXRXRXRXXYQFLI

SEQ ID NO: 17 - protein, 23 aa, synthetic construct
  MOD_RES 7: note = X is beta-Alanine
  MOD_RES 9: note = X is aminohexanoic acid
  MOD_RES 11: note = X is beta-Alanine
  MOD_RES 13: note = X is aminohexanoic acid
  MOD_RES 14: note = X is beta-Alanine
  MOD_RES 16: note = X is aminohexanoic acid
  MOD_RES 18: note = X is beta-Alanine
  source 1..23: note = peptide
YQFLIRXRXRXRXXRXRXYQFLI

SEQ ID NO: 18 - protein, 20 aa, synthetic construct
  MOD_RES 7: note = X is beta-Alanine
  MOD_RES 9: note = X is aminohexanoic acid
  MOD_RES 11: note = X is beta-Alanine
  MOD_RES 13: note = X is aminohexanoic acid
  MOD_RES 14: note = X is beta-Alanine
  source 1..20: note = peptide
YQFLIRXRXRXRXXRYQFLI

SEQ ID NO: 19 - protein, 24 aa, synthetic construct
  MOD_RES 7: note = X is beta-Alanine
  MOD_RES 10: note = X is aminohexanoic acid
  MOD_RES 12: note = X is beta-Alanine
  MOD_RES 14: note = X is aminohexanoic acid
  MOD_RES 15: note = X is beta-Alanine
  MOD_RES 17: note = X is aminohexanoic acid
  MOD_RES 19: note = X is beta-Alanine
  source 1..24: note = peptide
YQFLIRXRRXRXRXXRXRXYQFLI

SEQ ID NO: 20 - protein, 22 aa, synthetic construct
  MOD_RES 7: note = X is beta-Alanine
  MOD_RES 9: note = X is aminohexanoic acid
  MOD_RES 11: note = X is beta-Alanine
  MOD_RES 13: note = X is beta-Alanine
  MOD_RES 15: note = X is aminohexanoic acid
  MOD_RES 17: note = X is beta-Alanine
  source 1..22: note = peptide
YQFLIRXRXRXRXRXRXYQFLI

SEQ ID NO: 21 - protein, 22 aa, synthetic construct
  MOD_RES 7: note = X is beta-Alanine
  MOD_RES 9: note = X is aminohexanoic acid
  MOD_RES 11: note = X is beta-Alanine
  MOD_RES 13: note = X is beta-Alanine
  MOD_RES 15: note = X is beta-Alanine
  MOD_RES 17: note = X is beta-Alanine
  source 1..22: note = peptide
YQFLIRXRXRXRXRXRXYQFLI

SEQ ID NO: 22 - protein, 22 aa, synthetic construct
  MOD_RES 7: note = X is beta-Alanine
  MOD_RES 9: note = X is beta-Alanine
  MOD_RES 11: note = X is beta-Alanine
  MOD_RES 13: note = X is beta-Alanine
  MOD_RES 15: note = X is beta-Alanine
  MOD_RES 17: note = X is beta-Alanine
  source 1..22: note = peptide
YQFLIRXRXRXRXRXRXYQFLI

SEQ ID NO: 23 - protein, 21 aa, synthetic construct
  MOD_RES 7: note = X is beta-Alanine
  MOD_RES 9: note = X is aminohexanoic acid
  MOD_RES 11: note = X is beta-Alanine
  MOD_RES 13: note = X is beta-Alanine
  MOD_RES 15: note = X is aminohexanoic acid
  source 1..21: note = peptide
YQFLIRXRXRXRXRXRYQFLI

SEQ ID NO: 24 - protein, 22 aa, synthetic construct
  MOD_RES 7: note = X is beta-Alanine
  MOD_RES 10: note = X is beta-Alanine
  MOD_RES 12: note = X is beta-Alanine
  MOD_RES 14: note = X is beta-Alanine
  MOD_RES 17: note = X is beta-Alanine
  source 1..22: note = peptide
YQFLIRXRRXRXRXRRXYQFLI

SEQ ID NO: 25 - protein, 24 aa, synthetic construct
  MOD_RES 7: note = X is beta-Alanine
  MOD_RES 10: note = X is beta-Alanine
  MOD_RES 12: note = X is beta-Alanine
  MOD_RES 14: note = X is aminohexanoic acid
  MOD_RES 15: note = X is beta-Alanine
  MOD_RES 17: note = X is aminohexanoic acid
  MOD_RES 19: note = X is beta-Alanine
  source 1..24: note = peptide
YQFLIRXRRXRXRXXRXRXYQFLI

SEQ ID NO: 26 - protein, 24 aa, synthetic construct
  MOD_RES 7: note = X is beta-Alanine
  MOD_RES 10: note = X is beta-Alanine
  MOD_RES 12: note = X is beta-Alanine
  MOD_RES 14: note = X is beta-Alanine
  MOD_RES 15: note = X is beta-Alanine
  MOD_RES 17: note = X is aminohexanoic acid
  MOD_RES 19: note = X is beta-Alanine
  source 1..24: note = peptide
YQFLIRXRRXRXRXXRXRXYQFLI

SEQ ID NO: 27 - protein, 24 aa, synthetic construct
  MOD_RES 7: note = X is beta-Alanine
  MOD_RES 10: note = X is beta-Alanine
  MOD_RES 12: note = X is beta-Alanine
  MOD_RES 14: note = X is beta-Alanine
  MOD_RES 15: note = X is beta-Alanine
  MOD_RES 17: note = X is beta-Alanine
  MOD_RES 19: note = X is beta-Alanine
  source 1..24: note = peptide
YQFLIRXRRXRXRXXRXRXYQFLI

SEQ ID NO: 28 - protein, 24 aa, synthetic construct
  MOD_RES 7: note = X is beta-Alanine
  MOD_RES 10: note = X is beta-Alanine
  MOD_RES 12: note = X is beta-Alanine
  MOD_RES 14: note = X is beta-Alanine
  MOD_RES 15: note = X is beta-Alanine
  MOD_RES 17: note = X is beta-Alanine
  MOD_RES 19: note = X is beta-Alanine
  source 1..24: note = peptide
FQILYRXRRXRXRXXRXRXFQILY

SEQ ID NO: 29 - protein, 24 aa, synthetic construct
  MOD_RES 7: note = X is beta-Alanine
  MOD_RES 10: note = X is aminohexanoic acid
  MOD_RES 12: note = X is beta-Alanine
  MOD_RES 14: note = X is aminohexanoic acid
  MOD_RES 15: note = X is beta-Alanine
  MOD_RES 17: note = X is aminohexanoic acid
  MOD_RES 19: note = X is beta-Alanine
  source 1..24: note = peptide
YQFLIRXRRXRXRXXRXRXFQILY

SEQ ID NO: 30 - DNA, 25 bp, synthetic construct
  source 1..25: mol_type = other DNA; note = 25-mer PMO antisense sequence for mouse dystrophin exon-23
ggccaaacctcggcttacctgaaat

SEQ ID NO: 31 - DNA, 22 bp, synthetic construct
  source 1..22: mol_type = other DNA; note = Exon20Fo
cagaattctgccaattgctgag

SEQ ID NO: 32 - DNA, 20 bp, synthetic construct
  source 1..20: mol_type = other DNA; note = Exon26Ro
ttcttcagcttgtgtcatcc

SEQ ID NO: 33 - DNA, 24 bp, synthetic construct
  source 1..24: mol_type = other DNA; note = Exon20Fi
cccagtctaccaccctatcagagc

SEQ ID NO: 34 - DNA, 20 bp, synthetic construct
  source 1..20: mol_type = other DNA; note = Exon26Ri
cctgcctttaaggcttcctt

SEQ ID NO: 35 - DNA, 20 bp, synthetic construct
  source 1..20: mol_type = other DNA; note = mDMD23-24 primer 1
caggccattcctctttcagg

SEQ ID NO: 36 - DNA, 21 bp, synthetic construct
  source 1..21: mol_type = other DNA; note = mDMD23-24 primer 2
gaaactttcctcccagttggt

SEQ ID NO: 37 - DNA, 29 bp, synthetic construct
  modified_base 1: mod_base = OTHER; note = Labelled with FAM
  modified_base 9..10: mod_base = OTHER; note = Residues no. 9 and no. 10 are linked through an internal quencher ZEN
  modified_base 29: mod_base = OTHER; note = Labelled with IABkFQ
  source 1..29: mol_type = other DNA; note = mDMD23-24 probe
tcaacttcagccatccatttctgtaaggt

SEQ ID NO: 38 - DNA, 30 bp, synthetic construct
  source 1..30: mol_type = other DNA; note = mDMD22-24 primer 1
ctgaatatgaaataatggaggagagactcg

SEQ ID NO: 39 - DNA, 25 bp, synthetic construct
  source 1..25: mol_type = other DNA; note = mDMD22-24 primer 2
cttcagccatccatttctgtaaggt

SEQ ID NO: 40 - DNA, 19 bp, synthetic construct
  modified_base 1: mod_base = OTHER; note = Labelled with FAM
  modified_base 9..10: mod_base = OTHER; note = Residues no. 9 and no. 10 are linked through an internal quencher ZEN
  modified_base 19: mod_base = OTHER; note = Labelled with IABkFQ
  source 1..19: mol_type = other DNA; note = mDMD22-24 probe
atgtgattctgtaatttcc

(19) United States
(12) Patent Application Publication
(10) Pub. No.: US 2026/0091120 A1
(43) Pub. Date: Apr. 2, 2026

(54) CELL-PENETRATING PEPTIDES

(71) Applicants: Oxford University Innovation Limited, Oxford (GB); United Kingdom Research and Innovation, Swindon (GB)

(72) Inventors: Matthew WOOD, Oxford (GB); Samir EL ANDALOUSSI, Oxford (GB); Graham MCCLOREY, Oxford (GB); Raquel MANZANO, Zaragoza (ES); Michael J. GAIT, Cambridge (GB); Caroline GODFREY, Oxford (GB); Andrey ARZUMANOV, Cambridge (GB); Liz O'DONOVAN, Cork (IE); Richard RAZ, Copenhagen (DK)

(21) Appl. No.: 19/363,869

(22) Filed: Oct. 21, 2025

Related U.S. Application Data
(63) Continuation of application No. 17/266,940, now Pat. No. 12,472,264.

(30) Foreign Application Priority Data
Aug. 9, 2018 (GB) 1812980.9

(51) Int. Cl. A61K 47/64; C07K 7/08; C07K 14/00; C12N 15/113

(52) U.S. Cl. CPC A61K 47/645; C07K 7/08; C07K 14/00; C12N 15/113; C07K 2319/10; C12N 2310/11; C12N 2310/3513; C12N 2320/32

(57) ABSTRACT

The present invention relates to peptides, in particular cell-penetrating peptides, having a first hydrophobic domain positioned at the C-terminus of the peptide and a second hydrophobic domain positioned at the N-terminus of the peptide, and to conjugates of such cell-penetrating peptides with a therapeutic molecule. The present invention further relates to use of such peptides or conjugates in methods of treatment or as a medicament, especially in the treatment of genetic disorders and in particular muscular dystrophies such as Duchenne muscular dystrophy.

### CROSS-REFERENCE TO RELATED APPLICATIONS

This application is a continuation of U.S. application Ser. No. 17/266,940, filed Feb. 8, 2021, which is the U.S. national-stage of PCT Application No. PCT/GB2019/052248, filed Aug. 9, 2019, which claims the benefit of priority to United Kingdom Patent Application No. GB 1812980.9, filed Aug. 9, 2018, each of which is hereby incorporated by reference.

### SEQUENCE LISTING

The instant application contains a Sequence Listing which has been submitted electronically in XML format and is hereby incorporated by reference in its entirety. Said XML copy, created on Oct. 16, 2025, is named “51558-005003_Sequence_Listing_10_16_25” and is 106,363 bytes in size.

### TECHNICAL FIELD

The present invention relates to peptides, in particular cell-penetrating peptides, and to conjugates of such cell-penetrating peptides with a therapeutic molecule. The present invention further relates to use of such peptides or conjugates in methods of treatment or as a medicament, especially in the treatment of genetic disorders and in particular muscular dystrophies such as Duchenne muscular dystrophy.

### BACKGROUND

Nucleic acid drugs are genomic medicines with the potential to transform human healthcare. Research has indicated that such therapeutics could have applications across a broad range of disease areas including neuromuscular disease. The application of antisense oligonucleotide-based methods to modulate pre-mRNA splicing in the neuromuscular disuease Duchenne muscular dystrophy (DMD) has placed this monogenic disorder at the forefront of advances in precision medicine.

However, therapeutic development of these promising antisense therapeutics has been hampered by insufficient cell-penetrance and poor distribution characteristics—a challenge that is further emphasised by the large volume and dispersed nature of the muscle tissue substrate in DMD.

DMD affects one in 3500 new born boys. This severe, X-linked recessive disease results from mutations in the DMD gene that encodes dystrophin protein. The disorder is characterised by progressive muscle degeneration and wasting, along with the emergence of respiratory failure and cardiac complications, ultimately leading to premature death. The majority of mutations underlying DMD are genomic out-of-frame deletions that induce a premature truncation in the open reading frame that results in the absence of the dystrophin protein.

Exon skipping therapy utilises splice switching antisense oligonucleotides (SSOs) to target specific regions of the DMD transcript, inducing the exclusion of individual exons, leading to the restoration of aberrant reading frames and resulting in the production of an internally deleted, yet partially functional, dystrophin protein. Despite the undoubted potential of antisense oligonucleotide exon skipping therapy for DMD, the successful application of this appach is currently limited by the relatively inefficient targeting of skeletal muscle, as well as the inadequate targeting of single stranded oligonucleotides to other affected tissues such as the heart.

In September 2016 the Food and Drug Administration (FDA) granted accelerated approval for ‘eteplirsen’, a single stranded oligonucleotide for modulating the splicing of exon 51. Although this heralded the first approved oligonucleotide that modulates splicing in the US, the levels of dystrophin restoration were dissapointing with approximately 1% of normal dystrophin levels. Comparisons with the allelic disorder Becker muscular dystrophy and experiments in the mdx mouse have indicated that homogenous sarcolemmal dystrophin expression of at least ˜15% of wild-type is needed to protect muscle against exercise induced damage.

Therefore there is a strong and urgent need to improve the delivery of antisense oligonucleotides in order to provide a more effective therapy for devastating genetic diseases such as DMD.

The use of viruses as delivery vehicles has been suggested, however this is of limited use due to the immunotoxicity of the viral coat protein and potential oncogenic effects. Alternatively, a range of non-viral delivery vectors have been developed, amongst which peptides have shown the most promise due to their small size, targeting specificity and ability of trans-capillary delivery of large bio-cargoes. Several peptides have been reported for their ability to permeate cells either alone or carrying a bio-cargo.

For several years, cell-penetrating peptides (CPPs) have been conjugated to SSOs (in particular charge neutral phosphorodiamidate morpholino oligomer (PMO) and peptide nucleic acids (PNA)) to enhance the cell delivery by effectively carrying them across cell membranes to reach their pre-mRNA target sites in the cell nucleus. It has been shown that PMO therapeutics conjugated to certain arginine-rich CPPs (known as Peptide-PMOs or P-PMOs) can enhance dystrophin production in skeletal muscles following systemic administration in a mdx mouse model of DMD.

In particular, PNA/PMO internalization peptides (Pips) have been developed which are arginine rich CPPs, comprised of two arginine-rich sequences separated by a central short hydrophobic sequence. These ‘Pip’ peptides were designed to improve serum stability whilst maintaining a high level of exon skipping, initially by attachment to a PNA cargo. Further derivatives of these peptides were designed as conjugates of PMOs, which were shown to lead to body-wide skeletal muscle dystrophin production, and importantly also including the heart, following systemic administration in mice. Despite these peptides being efficacious, their therapeutic application has been hampered by the need for high dosage and therefore an associated toxicity.

Alternative cell-penetrating peptides having a single arginine rich domain such as R₆Gly have also been produced. These CPPs have been used to produce peptide conjugates with reduced toxicities, but in this case the conjugates exhibited low efficacy in comparison to the Pip peptides.

Accordingly, the currently available CPPs have not yet been demonstrated as suitable for use in human treatments for diseases such as DMD.

The challenge in the field of cell-penetrating peptide technology has been to improve efficacy without increasing toxicity. The present inventors have now identified, synthesized and tested a number of improved CPPs having a particular structure according to the present invention which address at least this problem.

These peptides maintain good levels of efficacy in skeletal muscles when tested in vitro and in vivo with a cargo therapeutic molecule. Furthermore, the peptides of the invention have improved levels of efficacy in skeletal muscles when tested in vitro and in vivo with a cargo therapeutic molecule. At the same time, these peptides when used in a conjugate allow therapeutic cargo molecules to penetrate into cells, and act effectively, at surprisingly lower doses than conjugates with previously available cell-penetrating peptides. The improved efficacy at lower doses means that the peptides of the invention do not have to be administered at high levels which can cause toxicity. Accordingly, the peptides of the invention offer improved suitability for use in therapeutic conjugates for the treatment of humans than previously available peptides.

### SUMMARY OF THE INVENTION

According to a first aspect of the present invention, there is provided a peptide having a total length of 40 amino acid residues or less, the peptide comprising:

One or more cationic domains; and

Two or more hydrophobic domains each comprising at least 3 amino acid residues;

wherein one of the hydrophobic domains is positioned at the C-terminus of the peptide and one of the hydrophobic domains is positioned at the N-terminus of the peptide.

According to a second aspect of the present invention, there is provided a conjugate comprising the peptide of the first aspect covalently linked to a therapeutic molecule.

According to a third aspect of the present invention, there is provided a conjugate comprising the peptide of the first aspect covalently linked to an imaging molecule.

According to a fourth aspect of the present invention, there is provided a pharmaceutical composition comprising the conjugate of the second aspect.

According to a fifth aspect of the present invention, there is provided a conjugate according to the second aspect for use as a medicament.

In one embodiment of the fifth aspect, there is provided a pharmaceutical composition according to the fourth aspect for use as a medicament.

According to a sixth aspect of the present invention, there is provided a method of treating a disease in a subject comprising administering the conjugate of the second aspect to the subject in a therapeutically effective amount.

In one embodiment of the sixth aspect, there is provided a method of treating a disease in a subject comprising administering the pharmaceutical composition according to the fourth aspect to the subject in a therapeutically effective amount.

According to a seventh aspect of the present invention, there is provided an isolated nucleic acid encoding the peptide of the first aspect or the conjugate of the second aspect or the conjugate of the third aspect.

According to an eighth aspect of the present invention, there is provided an expression vector comprising the nucleic acid sequence of the seventh aspect.

According to a ninth aspect of the present invention, there is provided a host cell comprising the expression vector of the eighth aspect.

### DETAILED DESCRIPTION

The inventors have produced a series of peptides that are suitable for use as cell-penetrating peptides to deliver therapeutic molecules into cells.

Surprisingly, the inventors have discovered a group of peptides having at least one cationic domain and at least two hydrophobic domains of defined lengths positioned at the N and C terminus of the peptide, which provide enhanced cell penetration into muscles, compared with currently available cell-penetrating peptides. This effect is observed when delivered as a conjugate with an antisense oligonucleotide therapeutic into cells, or when administered in vivo.

In the context of the disease DMD, enhanced cell penetration by peptides of the invention linked to a suitable therapeutic molecule can be shown by specific exon exclusion within the transcript. The directing of an antisense oligonucleotide to an appropriate sequence results in the forced skipping of an exon, the correction of the open reading frame and the restoration of an internally deleted, yet partially functional isoform of dystrophin.

The peptides of the present invention, when used as a conjugate with an antisense oligonucleotide therapeutic designed to target the dystrophin gene, are shown herein to have high levels of exon exclusion and dystrophin protein restoration.

In vivo, the results described herein show an increase in exon skipping and functional dystrophin expression when using peptide conjugates of the invention compared with the exon skipping results of the same antisense oligonucleotide therapeutic conjugated to a currently available cell-penetrating peptides. This is a significant improvement in the efficacy of such peptide carriers to penetrate muscle cells where neuromuscular diseases take effect.

Without wishing to be bound by theory, the inventors believe that the presence of terminal hydrophobic domains has the effect of boosting the delivery properties of the peptides to carry therapeutic cargo into cells. This is advantageous because increased cell penetration means increasing therapeutic molecule delivery and increased therapeutic effects. Furthermore these advantages can be provided at lower doses, thereby decreasing the likelihood of toxic effects of the cell-penetrating peptides.

It was completely unanticipated that such a peptide structure would increase the ability of an arginine-rich cell-penetrating peptide to transport a therapeutic molecule cargo, such as an oligonucleotide, into muscle.

It was unexpected that such transport would be enhanced so as to result in a therapeutic molecule such as an antisense oligonucleotide successfully increasing exon skipping and functional dystrophin protein production in various different muscles as demonstrated herein.

For the avoidance of doubt, and in order to clarify the way in which the present disclosure is to be interpreted, certain terms used in accordance with the present invention will now be defined further.

The invention includes any combination of the aspects and features described except where such a combination is clearly impermissible or expressly avoided.

The section headings used herein are for organisational purposes only and are not to be construed as limiting the subject matter described.

References to ‘X’ throughout denote any form of the non-natural amino acid aminohexanoic acid.

References to ‘B’ throughout denote the natural but non-genetically encoded amino acid residue beta-alanine.

References to ‘Ac’ throughout denote acetylation of the relevant peptide.

References to ‘Hyp’ throughout denote the natural but non-genetically encoded amino acid hydroxyproline.

References to other capital letters throughout denote the relevant genetically encoded amino acid residue in accordance with the accepted alphabetic amino acid code.

### Cationic Domain

The present invention relates to short cell-penetrating peptides having a particular structure in which there is at least one cationic domain.

References to ‘cationic’ herein denote an amino acid or domain of amino acids having an overall positive charge at physiological pH.

Suitably, the peptide comprises up to 4 cationic domains, up to 3 cationic domains, up to 2 cationic domains.

Suitably each cationic domain in the peptide may be identical or different. Suitably each cationic domain in the peptide is different.

Suitably, the peptide comprises 1 cationic domain.

Suitably, each cationic domain has a length of between 5 to 20 amino acid residues, suitably a length of between 9 to 14 amino acid residues.

Suitably, each cationic domain has a length of 9, 10, 11, 12, 13, or 14 amino acid residues.

Suitably, each cationic domain is of similar length, suitably each cationic domain is the same length.

Suitably, each cationic domain comprises cationic amino acids and may also contain polar and/or non-polar amino acids.

Non-polar amino acids may be selected from: alanine, beta-alanine, proline, glycine, cysteine, valine, leucine, isoleucine, methionine, tryptophan, phenylalanine. Suitably non-polar amino acids do not have a negative charge.

Polar amino acids may be selected from: serine, asparagine, hydroxyproline, histidine, arginine, threonine, tyrosine, glutamine. Suitably, the selected polar amino acids do not have a negative charge.

Cationic amino acids may be selected from: arginine, histidine, lysine. Suitably, cationic amino acids have a positive charge at physiological pH.

Suitably each cationic domain does not comprise anionic or negatively charged amino acid residues.

Suitably each cationic domain comprises arginine, aminohexanoic acid, histidine, beta-alanine, hydroxyproline and/or serine residues.

Suitably each cationic domain consists of arginine, aminohexanoic acid, and/or beta-alanine, residues.

Suitably, each cationic domain comprises at least 40%, at least 45%, at least 50% cationic amino acids.

Suitably, each cationic domain comprises a majority of cationic amino acids. Suitably, each cationic domain comprises at least 55%, at least 60%, at least 65% at least 70%, at least 75%, at least 80%, at least 85%, at least 90%, at least 95% cationic amino acids.

Suitably, each cationic domain comprises an isoelectric point (pl) of at least 7.5, at least 8.0, at least 8.5, at least 9.0, at least 9.5, at least 10.0, at least 10.5, at least 11.0, at least 11.5, at least 12.0.

Suitably, each cationic domain comprises an isoelectric point (pl) of at least 10.0.

Suitably, each cationic domain comprises an isoelectric point (pl) of between 11.0 and 13.0

In one embodiment, each cationic domain comprises an isoelectric point (pl) of between 12.3 and 12.7.

Suitably the isoelectric point of a cationic domain is calculated at physiological pH by any suitable means available in the art. Suitably, by using the IPC (www.isoelectric.org) a web-based algorithm developed by Lukasz Kozlowski, Biol Direct. 2016; 11:55. DOI: 10.1186/s13062-016-0159-9.

Suitably, each cationic domain comprises at least 4 cationic amino acids, suitably between 4-8 cationic amino acids.

Suitably, each cationic domain is arginine rich.

By ‘arginine rich’ it is meant that at least 40% of the cationic domain is formed of said residue.

Suitably, each cationic domain comprises a majority of arginine residues.

Suitably, a cationic domain may comprise at least 40%, at least 45%, at least 50%, at least 55%, at least 60%, at least 60%, at least 65%, least 70% arginine residues.

Suitably, a cationic domain may comprise a total of between 4-8 arginine residues. Suitably all of the cationic amino acids in a given cationic domain may be arginine.

Suitably, each cationic domain comprises one or more beta-alanine residues. Suitably, each cationic domain may comprise a total of between 3-7 beta-alanine residues, suitably a total of 4-6 beta-alanine residues.

Suitably, each cationic domain comprises one or more aminohexanoic acid residues. Suitably, each cationic domain may comprise a total of between 1-4 aminohexanoic acid residues, suitably a total of 1-3 aminohexanoic acid residues.

Suitably any aminohexanoic acid residue may be used, for example 4-aminohexanoic acid, 5-aminohexanoic acid, or 6-aminohexanoic acid. Suitably 6-aminohexanoic acid is used.

Suitably, a cationic domain may comprise one or more histidine, hydroxyproline residues or serine residues.

In one embodiment, the peptide comprises one arginine rich cationic domain.

Suitably, each cationic domain comprises no more than 3 contiguous arginine residues, no more than 2 contiguous arginine residues.

Suitably, each cationic domain may comprise arginine, aminohexanoic acid, and/or beta-alanine residues. Suitably, each cationic domain comprises a majority of arginine, aminohexanoic acid and/or beta-alanine residues. Suitably, at least 70%, at least 75%, at least 80%, at least 85%, at least 90%, at least 95%, 100% of the amino acid residues in each cationic domain are arginine, aminohexanoic acid and/or beta-alanine residues. Suitably, each cationic domain consists of arginine, aminohexanoic acid and/or beta-alanine residues.

Suitably, the peptide comprises one cationic domain, suitably this cationic domain forms the core of the peptide. Suitably the cationic domain is located at the centre of the peptide. Suitably therefore, the cationic domain may be known as the cationic core domain.

In one embodiment, the peptide comprises one cationic core domain that is arginine rich.

Suitably, each cationic domain comprises amino acid units selected from the following: R, X, B, RR, BB, XX, RX, XR, RB, BR, BX, XB, RBR, RBB, BBR, BRR, BRB, RBX, RXB, RBX, XRB, XRR, RRX, RXR, BXB, XBX, or any combination thereof.

Suitably, each cationic domain comprises any of the following sequences: RBRXRBRXB (SEQ ID NO:1), RBRXRBRXBRXRB (SEQ ID NO:2), RBRXRBRXBR (SEQ ID NO:3), RBRRXRBRXBRXRB (SEQ ID NO:4), RBRXRBRBRXRB (SEQ ID NO:5), RBRXRBRBRBRB (SEQ ID NO:6), RBRBRBRBRBRB (SEQ ID NO:7), RBRXRBRBRXR (SEQ ID NO:8), RBRRBRBRBRRB (SEQ ID NO:9), RBRRBRBRXBRXRB (SEQ ID NO:10), RBRRBRBRBBRXRB (SEQ ID NO:11), RBRRBRBRBBRBRB (SEQ ID NO:12), or any combination thereof.

Suitably, each cationic domain consists any of the following sequences: RBRXRBRXB (SEQ ID NO: 1), RBRXRBRXBRXRB (SEQ ID NO:2), RBRXRBRXBR (SEQ ID NO:3), RBRRXRBRXBRXRB (SEQ ID NO:4), RBRXRBRBRXRB (SEQ ID NO:5), RBRXRBRBRBRB

(SEQ ID NO:6), RBRBRBRBRBRB (SEQ ID NO:7), RBRXRBRBRXR (SEQ ID NO:8), RBRRBRBRBRRB (SEQ ID NO:9), RBRRBRBRXBRXRB (SEQ ID NO:10), RBRRBRBRBBRXRB (SEQ ID NO:11), RBRRBRBRBBRBRB (SEQ ID NO:12), or any combination thereof.

Suitably, each cationic domain consists of one of the following sequences: RBRRXRBRXBRXRB (SEQ ID NO:4), RBRRBRBRBRRB (SEQ ID NO:9), RBRRBRBRBBRXRB (SEQ ID NO:11).

Suitably each cationic domain in the peptide may be identical or different. Suitably each cationic domain in the peptide is different.

### Hydrophobic Domains

The present invention relates to short cell-penetrating peptides having a particular structure in which there are at least two hydrophobic domains having a certain length, wherein a hydrophobic domain is positioned at each terminal end of the peptide.

References to ‘hydrophobic’ herein denote an amino acid or domain of amino acids having the ability to repel water and/or which do not mix with water.

Suitably the peptide comprises up to 4 hydrophobic domains, up to 3 hydrophobic domains.

Suitably the peptide comprises 2 hydrophobic domains.

As defined above, the peptide comprises two or more hydrophobic domains each having a length of at least 3 amino acid residues.

Suitably, each hydrophobic domain has a length of between 3-6 amino acids. Suitably, each hydrophobic domain has a length of 5 amino acids.

Suitably, each hydrophobic domain may comprise non-polar, polar, and hydrophobic amino acid residues.

Hydrophobic amino acid residues may be selected from: alanine, valine, leucine, isoleucine, phenylalanine, tyrosine, methionine, and tryptophan.

Non-polar amino acid residues may be selected from: alanine, beta-alanine, valine, leucine, isoleucine, phenylalanine, methionine, tryptophan, proline, glycine, cysteine.

Polar amino acid residues may be selected from: serine, asparagine, hydroxyproline, histidine, arginine, threonine, tyrosine, glutamine.

Suitably the hydrophobic domains do not comprise hydrophilic amino acid residues.

Suitably, each hydrophobic domain comprises a majority of hydrophobic amino acid residues. Suitably each hydrophobic domain comprises at least 70%, at least 75%, at least 80%, at least 85%, at least 90%, at least 95%, 100% hydrophobic amino acids. Suitably, each hydrophobic domain consists of hydrophobic amino acid residues.

Suitably, each hydrophobic domain comprises a hydrophobicity of at least 0.3, at least 0.35, at least 0.4, at least 0.45.

Suitably, each hydrophobic domain comprises a hydrophobicity of between 0.4 and 1.4

In one embodiment, each hydrophobic domain comprises of a hydrophobicity of between 0.45 and 0.48.

Suitably, hydrophobicity is measured by White and Wimley: W. C. Wimley and S. H. White, “Experimentally determined hydrophobicity scale for proteins at membrane interfaces” Nature Struct Biol 3:842 (1996).

Suitably, each hydrophobic domain comprises at least 3, at least 4 hydrophobic amino acid residues.

Suitably, each hydrophobic domain comprises phenylalanine, leucine, isoleucine, tyrosine and glutamine residues. Suitably, each hydrophobic domain consists of phenylalanine, leucine, isoleucine, tyrosine and glutamine residues.

Suitably, the peptide comprises two hydrophobic domains. Suitably the hydrophobic domains are located at the N-terminus and C-terminus of the peptide, suitably at either end of the peptide. Suitably no further amino acids or domains are present at the N-terminus and C-terminus of the peptide, with the exception of other groups such as a terminal modification, linker and/or therapeutic molecule. For the avoidance of doubt, such other groups may be present in addition to ‘the peptide’ described and claimed herein. Suitably therefore each hydrophobic domain forms the terminus of the peptide. Suitably, this does not preclude the presence of a further linker group as described herein.

Suitably, the hydrophobic domains flank a core domain. Suitably, the hydrophobic domains may be considered as arm domains. Suitably the core domain may comprise one or more cationic domains and one or more further hydrophobic domains. Suitably the core domain comprises one cationic domain.

In one embodiment, the peptide comprises two hydrophobic arm domains flanking a core domain, wherein the core domain comprises one cationic domain.

In one embodiment, the peptide consists of two hydrophobic arm domains flanking a core domain, wherein the core domain comprises one cationic domain.

Suitably the or each hydrophobic domain comprises one of the following sequences: YQFLI (SEQ ID NO:13), FQILY (SEQ ID NO:14), ILFQY (SEQ ID NO:15), or any combination thereof.

Suitably the or each hydrophobic domain consists of one of the following sequences: YQFLI (SEQ ID NO:13), FQILY (SEQ ID NO:14), ILFQY (SEQ ID NO:15), or any combination thereof.

Suitably, the or each hydrophobic domain consists of the sequence YQFLI (SEQ ID NO:13).

Suitably each hydrophobic domain in the peptide may have the same sequence or a different sequence.

### Peptide

The present invention relates to short cell-penetrating peptides for use in transporting therapeutic cargo molecules in the treatment of medical conditions.

The peptide has a sequence that is a contiguous single molecule. Suitably, the peptide comprises several domains in a linear arrangement between the N-terminus and the C-terminus. Suitably, the domains are selected from cationic domains and hydrophobic domains described above. Suitably, the peptide consists of cationic domains and hydrophobic domains wherein the domains are as defined above.

Each domain has common sequence characteristics as described in the relevant sections above, but the exact sequence of each domain is capable of variation and modification. Thus a range of sequences is possible for each domain. The combination of each possible domain sequence yields a range of peptide structures, each of which form part of the present invention. Features of the peptide structures are described below.

Suitably, a cationic domain separates each hydrophobic domain. Suitably, each cationic domain is flanked by hydrophobic domains on either side thereof.

Suitably no hydrophobic domain is contiguous with another hydrophobic domain.

In one embodiment, the peptide comprises one cationic domain flanked by two hydrophobic domains in the following arrangement:

[hydrophobic domain]−[cationic domain]−[hydrophobic domain]

Therefore, suitably the cationic domain may be known as the core domain and each of the hydrophobic domains may be known as an arm domain. Suitably, the hydrophobic arm domains flank the cationic core domain on either side thereof.

In one embodiment, the peptide consists of one cationic domain and two hydrophobic domains.

In one embodiment, the peptide consists of one cationic core domain flanked by two hydrophobic arm domains.

In one embodiment, the peptide consists of one cationic core domain comprising a sequence selected from: RBRXRBRXB (SEQ ID NO:1), RBRXRBRXBRXRB (SEQ ID NO:2), RBRXRBRXBR (SEQ ID NO:3), RBRRXRBRXBRXRB (SEQ ID NO:4), RBRXRBRBRXRB (SEQ ID NO:5), RBRXRBRBRBRB (SEQ ID NO:6), RBRBRBRBRBRB (SEQ ID NO:7), RBRXRBRBRXR (SEQ ID NO:8), RBRRBRBRBRRB (SEQ ID NO:9), RBRRBRBRXBRXRB (SEQ ID NO:10), RBRRBRBRBBRXRB (SEQ ID NO:11), RBRRBRBRBBRBRB (SEQ ID NO: 12), flanked by two hydrophobic arm domains each comprising a sequence selected from: YQFLI (SEQ ID NO:13), FQILY (SEQ ID NO:14), ILFQY (SEQ ID NO:15).

In one embodiment, the peptide consists of one cationic core domain comprising a sequence selected from: RBRRXRBRXBRXRB (SEQ ID NO:4), RBRRBRBRBRRB (SEQ ID NO:9), RBRRBRBRBBRXRB (SEQ ID NO:11), flanked by two hydrophobic arm domains each comprising the sequence YQFLI (SEQ ID NO:13).

In any such embodiment, further groups may be present such as a linker, terminal modification and/or therapeutic molecule.

Suitably, the peptide is N-terminal modified.

Suitably, the peptide is N-acetylated, N-methylated, N-trifluoroacetylated, N-trifluoromethylsulfonylated, or N-methylsulfonylated. Suitably, the peptide is N-acetylated.

Optionally, the N-terminus of the peptide may be unmodified.

In one embodiment, the peptide is N-acetylated.

Suitably, the peptide is C-terminal modified.

Suitably, the peptide comprises a C-terminal modification selected from: Carboxy-, Thioacid-, Aminooxy-, Hydrazino-, thioester-, azide, strained alkyne, strained alkene, aldehyde-, thiol or haloacetyl-group.

Advantageously, the C-terminal modification provides a means for linkage of the peptide to the therapeutic molecule.

Accordingly, the C-terminal modification may comprise the linker and vice versa. Suitably, the C-terminal modification may consist of the linker or vice versa. Suitable linkers are described herein elsewhere.

Suitably, the peptide comprises a C-terminal carboxyl group.

Suitably, the C-terminal carboxyl group is provided by a glycine or beta-alanine residue.

In one embodiment, the C-terminal carboxyl group is provided by a beta-alanine residue.

Suitably, the C terminal beta-alanine residue is a linker.

Suitably, therefore each hydrophobic domain may further comprise an N or C terminal modification. Suitably the hydrophobic domain at the C terminus comprises a C-terminal modification. Suitably the hydrophobic domain at the N terminus comprises a N-terminal modification. Suitably, the hydrophobic domain at the C terminus comprises a linker group, suitably, the hydrophobic domain at the C terminus comprises a C-terminal beta-alanine. Suitably, the hydrophobic domain at the N terminus is N-acetylated.

The peptide of the present invention is defined as having a total length of 40 amino acid residues or less. The peptide may therefore be regarded as an oligopeptide.

Suitably, the peptide has a total length of between 10-35 amino acid residues, suitably of between 12-33 amino acid residues, of between 15-30 amino acid residues, of between 17-27 amino acid residues, of between 20-25 amino acid residues.

Suitably, the peptide has a total length of at least 16, at least 17, at least 18, at least 19, at least 20 amino acid residues.

Suitably the peptide is capable of penetrating cells. The peptide may therefore be regarded as a cell-penetrating peptide.

Suitably, the peptide is for attachment to a therapeutic molecule. Suitably, the peptide is for transporting a therapeutic molecule into a target cell. Suitably, the peptide is for delivering a therapeutic molecule into a target cell. The peptide may therefore be regarded as a carrier peptide.

Suitably the peptide is capable of penetrating into cells and tissues, suitably into the nucleus of cells. Suitably into muscle tissues.

Suitably, the peptide may be selected from any of the following sequences:

(SEQ ID NO: 16)
YQFLIRBRXRBRXBYQFLI
(SEQ ID NO: 17)
YQFLIRBRXRBRXBRXRBYQFLI
(SEQ ID NO: 18)
YQFLIRBRXRBRXBRYQFLI
(SEQ ID NO: 19)
YQFLIRBRRXRBRXBRXRBYQFLI
(SEQ ID NO: 20)
YQFLIRBRXRBRBRXRBYQFLI
(SEQ ID NO: 21)
YQFLIRBRXRBRBRBRBYQFLI
(SEQ ID NO: 22)
YQFLIRBRBRBRBRBRBYQFLI
(SEQ ID NO: 23)
YQFLIRBRXRBRBRXRYQFLI
(SEQ ID NO: 24)
YQFLIRBRRBRBRBRRBYQFLI
(SEQ ID NO: 25)
YQFLIRBRRBRBRXBRXRBYQFLI
(SEQ ID NO: 26)
YQFLIRBRRBRBRBBRXRBYQFLI
(SEQ ID NO: 27)
YQFLIRBRRBRBRBBRBRBYQFLI
(SEQ ID NO: 28)
FQILYRBRRBRBRBBRBRBFQILY
(SEQ ID NO: 29)
YQFLIRBRRXRBRXBRXRBFQILY

Suitably the peptide consists of one of the following sequences:

(SEQ ID NO: 19)
YQFLIRBRRXRBRXBRXRBYQFLI
(SEQ ID NO: 24)
YQFLIRBRRBRBRBRRBYQFLI
(SEQ ID NO: 26)
YQFLIRBRRBRBRBBRXRBYQFLI

In one embodiment, the peptide

(SEQ ID NO: 19)
YQFLIRBRRXRBRXBRXRBYQFLI

consists of the following sequence:

### Conjugate

The peptide of the invention may be covalently linked to a therapeutic molecule in order to provide a conjugate.

The therapeutic molecule may be any molecule for treatment of a disease. The therapeutic molecule may be selected from: a nucleic acid, peptide nucleic acid, antisense oligonucleotide (such as PNA, PMO), short interfering RNA, micro RNA, mRNA, gRNA (for example in the use of CRISPR/Cas9 technology), antagomiRNA, peptide, cyclic peptide, protein, pharmaceutical, drug or nanoparticle.

In one embodiment, the therapeutic molecule is an antisense oligonucleotide.

Suitably the antisense oligonucleotide is comprised of a phosphorodiamidate morpholino oligonucleotide (PMO).

Alternatively the oligonucleotide may be a modified PMO or any other charge-neutral oligonucleotide such as a peptide nucleic acid (PNA), a chemically modified PNA such as a gamma-PNA (Bahal, Nat.Comm. 2016), oligonucleotide phosphoramidate (where the non-bridging oxygen of the phosphate is substituted by an amine or alkylamine such as those described in WO2016028187A1, or any other partially or fully charge-neutralized oligonucleotide.

The therapeutic antisense oligonucleotide sequence may be selected from any that are available, for example antisense oligonucleotides for exon skipping in DMD are described inhttps://research-repository.uwa.edu.au/en/publications/antisense-oligonucleotide-induced-exon-skipping-across-the-human-, or a therapeutic antisense oligonucleotide complementary to the ISSN1 or IN7 sequence for the treatment of SMA are described in Zhou, H G T, 2013; and Hammond et al, 2016; and Osman et al, HMG, 2014.

Suitably, the antisense oligonucleotide sequence is for inducing exon skipping for use in the treatment of DMD.

Suitably, the antisense oligonucleotide sequence is for inducing exon skipping in the dystrophin gene for use in the treatment of DMD. Suitably the antisense oligonucleotide sequence may induce exon skipping of one or multiple exons.

In one embodiment, the antisense oligonucleotide sequence is for inducing exon skipping of a single exon of the dystrophin gene for use in the treatment of DMD. Suitably the single exon is selected from any exon implicated in DMD, which may be any exon in the dystrophin gene, such as for example, exon 45, 51 or 53. PMO oligonucleotides of any sequence may be purchased (for example from Gene Tools Inc, USA).

In one embodiment, the therapeutic molecule of the conjugate is an oligonucleotide complementary to the pre-mRNA of a gene target.

Suitably, the oligonucleotide complementary to the pre-mRNA of a gene target gives rise to a steric blocking event that alters the pre-mRNA leading to an altered mRNA and hence a protein of altered sequence. Suitably, the gene target is the dystrophin gene.

Suitably the steric blocking event may be exon inclusion or exon skipping. In one embodiment, the steric blocking event is exon skipping, suitably exon skipping of a single exon of the dystrophin gene.

Optionally, lysine residues may be added to one or both ends of a therapeutic molecule (such as a PMO or PNA) before attachment to the peptide to improve water solubility.

Suitably the therapeutic molecule has a molecular weight of less than 5,000 Da, suitably less than 3,000 Da or suitably less than 1,000 Da.

Suitably, the peptide is covalently linked to the therapeutic molecule at the C-terminus.

Suitably, the peptide is covalently linked to the therapeutic molecule through a linker if required. The linker may act as a spacer to separate the peptide sequence from the therapeutic molecule. In one embodiment, the conjugate comprises a linker.

The linker may be selected from any suitable sequence.

Suitably, the linker may be part of the peptide or the therapeutic molecule. Suitably the linker is present between the peptide and the therapeutic molecule.

In one embodiment, the conjugate comprises the peptide covalently linked via a linker to a therapeutic molecule. In one embodiment, the conjugate comprises the following structure:

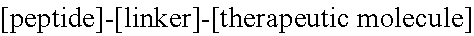

In one embodiment, the conjugate consists of the following structure:

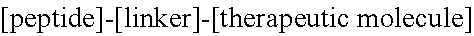

Suitably any of the peptides listed herein may be used in a conjugate according to the invention. In one embodiment, the conjugate comprises a peptide selected from one of the following sequences: YQFLIRBRRXRBRXBRXRBYQFLI (SEQ ID NO:19), YQFLIRBRRBRBRBRRBYQFLI (SEQ ID NO:24), and YQFLIRBRRBRBRBBRXRBYQFLI (SEQ ID NO:26).

Suitably, in any case, the peptide may further comprise N-terminal modifications as described above.

Suitable linkers include, for example, a C-terminal cysteine residue that permits formation of a disulphide, thioether or thiol-maleimide linkage, a C-terminal aldehyde to form an oxime, a click reaction or formation of a morpholino linkage with a basic amino acid on the peptide or a carboxylic acid moiety on the peptide covalently conjugated to an amino group to form a carboxamide linkage.

Suitably, the linker is between 1-5 amino acids in length. Suitably the linker may comprise any linker that is known in the art.

Suitably the linker is selected from any of the following sequences: G, BC, XC, C, GGC, BBC, BXC, XBC, X, XX, B, BB, BX and XB.

Suitably, the linker may be a polymer, such as for example PEG.

In one embodiment, the linker is beta-alanine.

In one embodiment, the peptide is conjugated to the therapeutic molecule through a carboxamide linkage.

The linker of the conjugate may form part of the therapeutic molecule to which the peptide is attached. Alternatively, the attachment of the therapeutic molecule may be directly linked to the C-terminus of the peptide. Suitably, in such embodiments, no linker is required.

Alternatively, the peptide may be chemically conjugated to the therapeutic molecule. Chemical linkage may be via a disulphide, alkenyl, alkynyl, aryl, ether, thioether, triazole, amide, carboxamide, urea, thiourea, semicarbazide, carbazide, hydrazine, oxime, phosphate, phosphoramidate, thiophosphate, boranophosphate, iminophosphates, or thiol-maleimide linkage, for example.

Optionally, cysteine may be added at the N-terminus of a therapeutic molecule to allow for disulphide bond formation to the peptide, or the N-terminus may undergo bromoacetylation for thioether conjugation to the peptide.

The peptide of the invention may equally be covalently linked to an imaging molecule in order to provide a conjugate.

Suitably, the imaging molecule may be any molecule that enables visualisation of the conjugate. Suitably, the imaging molecule may indicate the location of the conjugate. Suitably the location of the conjugate in vitro or in vivo. Suitably, there is provided a method of monitoring the location of a conjugate comprising an imaging molecule comprising: administering the conjugate to a subject and imaging the subject to locate the conjugate.

Examples of imaging molecules include detection molecules, contrast molecules, or enhancing molecules. Suitable imaging molecules may be selected from radionuclides; fluorophores; nanoparticles (such as a nanoshell); nanocages; chromogenic agents (for example an enzyme), radioisotopes, dyes, radioopaque materials, fluorescent compounds, and combinations thereof.

Suitably imaging molecules are visualised using imaging techniques, these may be cellular imaging techniques or medical imaging techniques. Suitable cellular imaging techniques include image cytometry, fluorescent microscopy, phase contrast microscopy, SEM, TEM, for example. Suitable medical imaging techniques include X-ray, fluoroscopy, MRI, scintigraphy, SPECT, PET, CT, CAT, FNRI, for example.

In some cases, the imaging molecule may be regarded as a diagnostic molecule. Suitably, a diagnostic molecule enables the diagnosis of a disease using the conjugate. Suitably, diagnosis of a disease may be achieved through determining the location of the conjugate using an imaging molecule. Suitably, there is provided a method of diagnosis of a disease comprising administering an effective amount of a conjugate comprising an imaging molecule to a subject and monitoring the location of the conjugate.

Suitably, further details such as the linkage of a conjugate comprising an imaging molecule are the same as those described above in relation to a conjugate comprising a therapeutic molecule.

Suitably, the peptide of the invention may be covalently linked to a therapeutic molecule and an imaging molecule in order to provide a conjugate.

Suitably the conjugate is capable of penetrating into cells and tissues, suitably into the nucleus of cells. Suitably into muscle tissues.

### Pharmaceutical Composition

The conjugate of the invention may formulated into a pharmaceutical composition.

Suitably the pharmaceutical composition comprises a conjugate of the invention.

Suitably, the pharmaceutical composition may further comprise a pharmaceutically acceptable diluent, adjuvant or carrier.

Suitable pharmaceutically acceptable diluents, adjuvants and carriers are well known in the art.

As used herein, the phrase “pharmaceutically acceptable” refers to those ligands, materials, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier”, as used herein, refers to a pharmaceutically acceptable material, formulation or vehicle, such as a liquid or solid filler, diluent, excipient, solvent or encapsulating material, involved in carrying or transporting the conjugate from one organ or portion of the body, to another organ or portion of the body. Each cell-penetrating peptide must be “acceptable” in the sense of being compatible with the other components of the composition e.g. the peptide and therapeutic molecule, and not injurious to the individual. Lyophilized compositions, which may be reconstituted and administered, are also within the scope of the present composition.

Pharmaceutically acceptable carriers may be, for example, excipients, vehicles, diluents, and combinations thereof. For example, where the compositions are to be administered orally, they may be formulated as tablets, capsules, granules, powders, or syrups; or for parenteral administration, they may be formulated as injections, drop infusion preparations, or suppositories. These compositions can be prepared by conventional means, and, if desired, the active compound (i.e. conjugate) may be mixed with any conventional additive, such as an excipient, a binder, a disintegrating agent, a lubricant, a corrigent, a solubilizing agent, a suspension aid, an emulsifying agent, a coating agent, or combinations thereof.

It should be understood that the pharmaceutical compositions of the present disclosure can further include additional known therapeutic agents, drugs, modifications of compounds into prodrugs, and the like for alleviating, mediating, preventing, and treating the diseases, disorders, and conditions described herein under medical use. Suitably, the pharmaceutical composition is for use as a medicament. Suitably for use as a medicament in the same manner as described herein for the conjugate. All features described herein in relation to medical treatment using the conjugate apply to the pharmaceutical composition.

Accordingly, in a further aspect of the invention there is provided a pharmaceutical composition according to the fourth aspect for use as a medicament. In a further aspect, there is provided a method of treating a subject for a disease condition comprising administering an effective amount of a pharmaceutical composition according to the fourth aspect to the subject.

### Medical Use

The conjugate comprising the peptide of the invention may be used as a medicament for the treatment of a disease.

The medicament may be in the form of a pharmaceutical composition as defined above.

A method of treatment of a patient or subject in need of treatment for a disease condition is also provided, the method comprising the step of administering a therapeutically effective amount of the conjugate to the patient or subject.

Suitably, the medical treatment requires delivery of the therapeutic molecule into a cell, suitably into the nucleus of the cell.

Diseases to be treated may include any disease where improved penetration of the cell and/or nuclear membrane by a therapeutic molecule may lead to an improved therapeutic effect.

Suitably, the conjugate is for use in the treatment of diseases of the neuromuscular system.

Conjugates comprising peptides of the invention are suitable for the treatment of genetic diseases of the neuromuscular system. Conjugates comprising peptides of the invention are suitable for the treatment of genetic neuromuscular diseases. In a suitable embodiment, there is provided a conjugate according to the second aspect for use in the treatment of genetic diseases of the neuromuscular system. Suitably, the conjugate is for use in the treatment of hereditary genetic diseases. Suitably, the conjugate is for use in the treatment of hereditary genetic neuromuscular diseases. Suitably, the conjugate is for use in the treatment of hereditary genetic neuromuscular diseases. Suitably, the conjugate is for use in the treatment of hereditary X-linked genetic diseases of the neuromuscular system. Suitably, the conjugate is for use in the treatment of hereditary X-linked neuromuscular diseases.

Suitably, the conjugate is for use in the treatment of diseases caused by splicing deficiencies. In such embodiments, the therapeutic molecule may comprise an oligonucleotide capable of preventing or correcting the splicing defect and/or increasing the production of correctly spliced mRNA molecules.

Suitably the conjugate is for use in the treatment of any of the following diseases: Duchenne Muscular Dystrophy (DMD), Bucher Muscular Dystrophy (BMD), Menkes disease, Beta-thalassemia, dementia, Parkinson's Disease, Spinal Muscular Atrophy (SMA), myotonic dystrophy (DM), Huntington's Disease, Hutchinson-Gilford Progeria Syndrome, Ataxia-telangiectasia, or cancer.

In one embodiment, the conjugate is for use in the treatment of DMD.

In one embodiment, there is provided a conjugate according to the second aspect for use in the treatment of DMD.

Suitably, in such an embodiment, the therapeutic molecule of the conjugate is operable to increase expression of the dystrophin protein. Suitably, in such an embodiment, the therapeutic molecule of the conjugate is operable to increase the expression of functional dystrophin protein.

Suitably the conjugate increases dystrophin expression by 10%, 15%, 20%, 25%, 30%, 35%, 40%, 45%, 50%, 55%, 60%, 65%, 70%. Suitably the conjugate increases dystrophin expression by up to 50%.

Suitably the conjugate restores dystrophin protein expression by 10%, 15%, 20%, 25%, 30%, 35%, 40%, 45%, 50%, 55%, 60%, 65%, 70%. Suitably the conjugate restores dystrophin protein expression by up to 50%.

Suitably the conjugate restores dystrophin protein function by 10%, 15%, 20%, 25%, 30%, 35%, 40%, 45%, 50%, 55%, 60%, 65%, 70%. Suitably the conjugate restores dystrophin protein function by up to 50%.

Suitably, the therapeutic molecule of the conjugate is operable to do so by causing skipping of one or more exons during dystrophin transcription.

Suitably, the conjugate causes 10%, 15%, 20%, 25%, 30%, 35%, 40%, 45%, 50%, 55%, 60%, 65%, 70%, 75%, 80%, 85% skipping of one or more exons of the dystrophin gene. Suitably, the conjugate causes up to 50% skipping of one or more exons of the dystrophin gene.

Suitably, the patient or subject to be treated may be any animal or human. Suitably, the patient or subject may be a non-human mammal. Suitably the patient or subject may be male or female. In one embodiment, the subject is male.

Suitably, the patient or subject to be treated may be any age. Suitably the patient or subject to be treated is aged between 0-40 years, suitably 0-30, suitably 0-25, suitably 0-20 years of age.

Suitably, the conjugate is for administration to a subject systemically for example by intramedullary, intrathecal, intraventricular, intravitreal, enteral, parenteral, intravenous, intra-arterial, intramuscular, intratumoural, subcutaneous, oral or nasal routes.

In one embodiment, the conjugate is for administration to a subject intravenously. In one embodiment, the conjugate is for administration to a subject intravenously by injection.

Suitably, the conjugate is for administration to a subject in a “therapeutically effective amount”, by which it is meant that the amount is sufficient to show benefit to the individual. The actual amount administered, and rate and time-course of administration, will depend on the nature and severity of the disease being treated. Decisions on dosage are within the responsibility of general practitioners and other medical doctors. Examples of the techniques and protocols can be found in Remington's Pharmaceutical Sciences, 20th Edition, 2000, pub. Lippincott, Williams & Wilkins.

Exemplary doses may be between 0.01 mg/kg and 50 mg/kg, 0.05 mg/kg and 40 mg/kg, 0.1 mg/kg and 30 mg/kg, 0.5 mg/kg and 18 mg/kg, 1 mg/kg and 16 mg/kg, 2 mg/kg and 15 mg/kg, 5 mg/kg and 10 mg/kg, 10 mg/kg and 20 mg/kg, 12 mg/kg and 18 mg/kg, 13 mg/kg and 17 mg/kg. Advantageously, the dosage of the conjugates of the present invention is an order or magnitude lower than the dosage required to see any effect from the therapeutic molecule alone, and lower than currently available cell-penetrating peptide conjugates.

### Nucleic Acids and Hosts

Peptides of the invention may be produced by any standard protein synthesis method, for example chemical synthesis, semi-chemical synthesis or through the use of expression systems.

Accordingly, the present invention also relates to the nucleotide sequences comprising or consisting of the DNA coding for the peptides, expression systems e.g. vectors comprising said sequences accompanied by the necessary sequences for expression and control of expression, and host cells and host organisms transformed by said expression systems.

Accordingly, a nucleic acid encoding a peptide according to the present invention is also provided.

Suitably, the nucleic acids may be provided in isolated or purified form.

An expression vector comprising a nucleic acid encoding a peptide according to the present invention is also provided.

Suitably, the vector is a plasmid.

Suitably the vector comprises a regulatory sequence, e.g. promoter, operably linked to a nucleic acid encoding a peptide according to the present invention. Suitably, the expression vector is capable of expressing the peptide when transfected into a suitable cell, e.g. mammalian, bacterial or fungal cell.

A host cell comprising the expression vector of the invention is also provided.

Expression vectors may be selected depending on the host cell into which the nucleic acids of the invention may be inserted. Such transformation of the host cell involves conventional techniques such as those taught in Sambrook et al [Sambrook, J., Russell, D. (2001) Molecular Cloning: A Laboratory Manual, Cold Spring Harbor Laboratory Press, NY, USA]. Selection of suitable vectors is within the skills of the person knowledgeable in the field. Suitable vectors include plasmids, bacteriophages, cosmids, and viruses.

The peptides produced may be isolated and purified from the host cell by any suitable method e.g. precipitation or chromatographic separation e.g. affinity chromatography.

Suitable vectors, hosts and recombinant techniques are well known in the art.

In this specification the term “operably linked” may include the situation where a selected nucleotide sequence and regulatory nucleotide sequence are covalently linked in such a way as to place the expression of a nucleotide coding sequence under the control of the regulatory sequence, as such, the regulatory sequence is capable of effecting transcription of a nucleotide coding sequence which forms part or all of the selected nucleotide sequence.

Where appropriate, the resulting transcript may then be translated into a desired peptide.

### BRIEF DESCRIPTION OF THE DRAWINGS

Certain embodiments of the present invention will now be described with reference to the following figures and tables in which:

FIG. 1: shows the in vitro exon 23 skipping efficacy of some of the DPEP2 series of peptides conjugated to an antisense therapeutic molecule at 0.25 μM, 0.5 μM and 1 μM in H2K-mdx cells as measured by densitometry analysis of nested RT-PCR (Error bars: standard deviation, n≥3);

FIG. 2: shows the in vivo efficacy of some of the DPEP2 series of peptides conjugated to an antisense therapeutic molecule in (A) Tibalis anterior muscle, (B) diaphragm, and (C) heart muscle in comparison with currently available peptides conjugated to the same antisense therapeutic molecule following a single 10 mg/kg intravenous dose into mdx mice, dystrophin protein restoration was assessed by western blot and exon skipping activity by qRTPCR (Error bars: standard deviation, n=3).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The reader's attention is directed to all papers and documents which are filed concurrently with or previous to this specification in connection with this application and which are open to public inspection with this specification, and the contents of all such papers and documents are incorporated herein by reference.

### EXAMPLES

### 1. Materials and Methods

### 1.1 P-PMO Synthesis and Preparation

9-Fluroenylmethoxycarbonyl (Fmoc) protected L-amino acids, benzotriazole-1-yl-oxy-tris-pyrrolidino-phosphonium (PyBOP), 2-(1H-benzotriazole-1-yl)-1,1,3,3-tetramethyluronium hexafluorophosphate (HBTU), and the Fmoc-β-Ala-OH preloaded Wang resin (0.19 or 0.46 mmol g⁻¹) were obtained from Merck (Hohenbrunn, Germany). HPLC grade acetonitrile, methanol and synthesis grade N-methyl-2-pyrrolidone (NMP) were purchased from Fisher Scientific (Loughborough, UK). Peptide synthesis grade N,N-dimethylformamide (DMF) and diethyl ether were obtained from VWR (Leicestershire, UK). Piperidine and trifluoroacetic acid (TFA) were obtained from Alfa Aesar (Heysham, England). PMO was purchased from Gene Tools Inc. (Philomath, USA). Chicken Embryo Extract and horse serum were obtained from Sera Laboratories International Ltd (West Sussex, UK). Interferon was obtained from Roche Applied Science (Penzberg, Germany). All other reagents were obtained from Sigma-Aldrich (St. Louis, MO, USA) unless otherwise stated. MALDI-TOF mass spectrometry was carried out using a Voyager DE Pro BioSpectrometry workstation. A stock solution of 10 mg mL-1 of α-cyano-4-hydroxycinnamic acid or sinapinic acid in 50% acetonitrile in water was used as matrix. Error bars are ±0.1%.

### 1.2 Synthesis of P-PMO Peptides for Screening in H2k Mdx Cells

### a) Preparation of a Library of Peptide Variants

Peptides were either prepared on a 10 μmol scale using an Intavis Parallel Peptide Synthesizer or on a 100 μmol scale using a CEM Liberty Blue™ Peptide Synthesizer (Buckingham, UK) using Fmoc-β-Ala-OH preloaded Wang resin (0.19 or 0.46 mmol g⁻¹, Merck Millipore) by applying standard Fmoc chemistry and following manufacturer's recommendations. In the case of synthesis using the Intavis Parallel Peptide Synthesizer, double coupling steps were used with a PyBOP/NMM coupling mixture followed by acetic anhydride capping after each step. For synthesis using the CEM Liberty Blue Peptide Synthesizer, single standard couplings were implemented for all amino acids except arginine, which was performed by double couplings. The coupling was carried out once at 75° C. for 5 min at 60-watt microwave power except for arginine residues, which were coupled twice each. Each deprotection reaction was carried out at 75° C. twice, once for 30 sec and then for 3 min at 35-watt microwave power. Once synthesis was complete, the resin was washed with DMF (3×50 mL) and the N-terminus of the solid phase bound peptide was acetylated with acetic anhydride in the presence of DIPEA. at room temperature. After acetylation of the N-terminus, the peptide resin was washed with DMF (3×20 mL) and DCM (3×20 mL). The peptides were cleaved from the solid support by treatment with a cleavage cocktail consisting of trifluoroacetic acid (TFA): H₂O: triisopropylsilane (TIPS) (95%: 2.5%: 2.5%: 3-10 mL) for 3 h at room temperature. After peptide release, excess TFA was removed by sparging with nitrogen. The crude peptide was precipitated by the addition of cold diethyl ether (15-40 mL depending on scale of the synthesis) and centrifuged at 3200 rpm for 5 min. The crude peptide pellet was washed thrice with cold diethyl ether (3×15 mL) and purified by RP-HPLC using a Varian 940-LC HPLC System fitted with a 445-LC Scale-up module and 440-LC fraction collector. Peptides were purified by semi-preparative HPLC on an RP-C18 column (10×250 mm, Phenomenex Jupiter) using a linear gradient of CH₃CN in 0.1% TFA/H₂O with a flow rate of 15 mL min 1. Detection was performed at 220 nm and 260 nm. The fractions containing the desired peptide were combined and lyophilized to yield the peptide as a white solid (see Table 2 for yields).

TABLE 1
Peptides synthesised for the examples with N-terminal
acetylation and C-terminal beta-alanine linker, X is
6-aminohexanoic acid.

| | Sequence ID | Peptide sequence (including N |
|---|---|---|
| PepGen | NO. | terminal and C terminal |
| Number | Incorporated | modifications) |
| D-PEP 2.1 | 16 | Ac-YQFLIRBRXRBRXBYQFLI-B |
| D-PEP 2.2 | 17 | Ac-YQFLIRBRXRBRXBRXRBYQFLI-B |
| D-PEP 2.3 | 18 | Ac-YQFLIRBRXRBRXBRYQFLI-B |
| D-PEP 2.4 | 19 | Ac-YQFLIRBRRXRBRXBRXRBYQFLI-B |
| D-PEP 2.5 | 20 | Ac-YQFLIRBRXRBRBRXRBYQFLI-B |
| D-PEP 2.6 | 21 | Ac-YQFLIRBRXRBRBRBRBYQFLI-B |
| D-PEP 2.7 | 22 | Ac-YQFLIRBRBRBRBRBRBYQFLI-B |
| D-PEP 2.8 | 23 | Ac-YQFLIRBRXRBRBRXRYQFLI-B |
| D-PEP 2.9 | 24 | Ac-YQFLIRBRRBRBRBRRBYQFLI-B |
| D-PEP 2.10 | 25 | Ac-YQFLIRBRRBRBRXBRXRBYQFLI-B |
| D-PEP 2.11 | 26 | Ac-YQFLIRBRRBRBRBBRXRBYQFLI-B |
| D-PEP 2.12 | 27 | Ac-YQFLIRBRRBRBRBBRBRBYQFLI-B |
| D-PEP 2.13 | 28 | Ac-FQILYRBRRBRBRBBRBRBFQILY-B |
| D-PEP 2.14 | 29 | Ac-YQFLIRBRRXRBRXBRXRBFQILY- |

TABLE 2
Yields of peptides synthesized for conjugation.

| PepGen | |
|---|---|
| Number | Yield |
| D-PEP 2.1 | 74% |
| D-PEP 2.2 | 63% |
| D-PEP 2.3 | 71% |
| D-PEP 2.4 | 75% |
| D-PEP 2.5 | 73% |
| D-PEP 2.6 | 69% |
| D-PEP 2.7 | 65% |
| D-PEP 2.8 | 59% |
| D-PEP 2.9 | 72% |
| D-PEP 2.10 | 65% |
| D-PEP 2.11 | 62% |
| D-PEP 2.12 | 51% |
| D-PEP 2.13 | 73% |
| D-PEP 2.14 | 67% |

### b) Synthesis of a Library of PMO-Peptide Conjugates

A 25-mer PMO antisense sequence for mouse dystrophin exon-23 (GGCCAAACCTCGGCTTACCTGAAAT (SEQ ID NO:30)) was used. The peptide was conjugated to the 3′-end of the PMO through its C-terminal carboxyl group. This was achieved using 2.5 and 2 equivalents of PyBOP and HOAt in NMP respectively in the presence of 2.5 equivalents of DIPEA and 2.5 fold excess of peptide over PMO dissolved in DMSO was used. In a few examples, 2 equivalents of HBTU were used in place of PyBOP for activation of the C-terminal carboxyl group of the peptide. In general, to a solution of peptide (2500 nmol) in N-methylpyrrolidone (NMP, 80 μL) were added PyBOP (19.2 μL of 0.3 M in NMP), HOAt in (16.7 μL of 0.3 M NMP), DIPEA (1.0 μL) and PMO (100 μL of 10 mM in DMSO). The mixture was left for 2.5 h at 40° C. and the reaction was quenched by the addition of 0.1% TFA in H₂O (300 μL). This solution was purified by Ion exchange chromatography using a converted Gilson HPLC system. The PMO-peptide conjugates were purified on an ion exchange column (Resource S 4 mL, GE Healthcare) using a linear gradient of sodium phosphate buffer (25 mM, pH 7.0) containing 20% CH₃CN. A sodium chloride solution (1 M) was used to elute the conjugate from the column at a flow rate of 4 mL min-1. The fractions containing the desired compound were combined and lyophilized to yield the peptide-PMO derivative as a white solid. The removal of excess salts from the peptide-PMO conjugate was afforded through the filtration of the fractions collected after ion exchange using an Amicon® ultra-15 3K centrifugal filter device. The conjugate was lyophilized and analyzed by MALDI-TOF. The conjugates were dissolved in sterile water and filtered through a 0.22 μm cellulose acetate membrane before use. The concentration of peptide-PMO was determined by the molar absorption of the conjugates at 265 nm in 0.1 N HCl solution. (see Table 3 for yields).

TABLE 3
Yields of P-PMO conjugates for cell culture analysis (The yields are
based on dried weight of the lyophilised purified ppmo. The purity
for the P-PMOs is greater than 95% as ascertained by normal phase
HPLC at 220 nm and 260 nm.

| Peptide Conjugate | Yield |
|---|---|
| D-PEP 2.1-PMO | 62% |
| D-PEP 2.2-PMO | 48% |
| D-PEP 2.3-PMO | 47% |
| D-PEP 2.4-PMO | 65% |
| D-PEP 2.5-PMO | 62% |
| D-PEP 2.6-PMO | 58% |
| D-PEP 2.7-PMO | 54% |
| D-PEP 2.8-PMO | 63% |
| D-PEP 2.9-PMO | 65% |
| D-PEP 2.10-PMO | 49% |
| D-PEP 2.11-PMO | 62% |
| D-PEP 2.12-PMO | 66% |
| D-PEP 2.13-PMO | 68% |
| D-PEP 2.14-PMO | 53% |

### 1.3 Cell Culture

Murine H2k mdx myoblasts were cultured in gelatin (0.01%) coated flasks at 33° C., under 10% CO₂ in Dulbecco's modified Eagles medium (DMEM PAA laboratories) supplemented with 20% heat-inactivated fetal bovine serum (FBS Gold, PAA laboratories), 2% chicken embryo extract (Seralab), 1% penicillin-streptomycin-neomycin antibiotic mixture (PSN, Gibco) and 3 pg/μL γ-interferon (Roche). Cells were seeded in gelatin (0.01%) coated 24-well plates at a density of 2×10⁵ cell/mL and left for 2 days at 33° C., 10% CO₂. To differentiate into myotubes, cells were further grown in DMEM supplemented with 5% horse serum (Sigma) and 1% PSN at 37° C., under 5% CO₂ for 2 days.

### 1.4 Cell Transfection

Cells were incubated with peptide-PMO conjugates prepared as described above which were made up in serum-free Opti-MEM and 350 μL was added to each well as duplicates and incubated at 37° C. for 4 hr. The transfection medium was then replaced with DMEM supplemented with 5% horse serum and 1% PSN and the cells incubated for a further 20 hr at 37° C. Cells were washed with PBS and 0.5 mL of TRI RNA (Sigma) isolation reagent was added to each well. Cells were frozen at −80° C. for 1 hr.

### 1.5 RNA Extraction and Nested RT-PCR Analysis

Total cellular RNA was extracted using TRI reagent with an extra further precipitation with ethanol. The purified RNA was quantified using a Nanodrop® ND-1000 (Thermo Scientific).

The RNA (400 ng) was used as a template for RT-PCR using a OneStep RT-PCR Kit (Roche, Indianapolis, USA). For primer sequences refer to Table 5. The cycle conditions for the initial reverse transcription were 50° C. for 30 min and 94° C. for 7 min for 1 cycle followed by 30 cycles of 94° C. for 20 sec, 55° C. for 40 sec and 68° C. for 80 sec. One microlitre of the RT-PCR product was used as template for the second PCR step. The amplification was carried out using 0.5 U of SuperTAQ in 25 cycles at 94° C. for 30 sec, 55° C. for 1 min and 72° C. for 1 min. the products were separated by electrophoresis using 1.5% agarose gel. The images of agarose gels were taken on a Molecular Imager ChemiDoc™ XRS⁺ imaging system (BioRad, UK) and the images were analysed using Image Lab (V4.1). Microsoft Excel was used to analyse and plot the exon-skipping assay data, which were expressed as the percentage of exon-23 skipping from at least three independent experiments.

### 1.6 Synthesis of PMO-Peptide Conjugates for Testing in H2k Mdx Mice

### a) Synthesis of Peptide Variants

Peptides were synthesized on a 100 μmol scale using a CEM Liberty Blue™ microwave Peptide Synthesizer (Buckingham, UK) and Fmoc chemistry following manufacturer's recommendations. The side chain protecting groups used were labile to trifluoroacetic acid treatment and the peptide was synthesized using a 5-fold excess of Fmoc-protected amino acids (0.25 mmol) that were activated using PyBOP (5-fold excess) in the presence of DIPEA. Piperidine (20% v/v in DMF) was used to remove N-Fmoc protecting groups. The coupling was carried out once at 75° C. for 5 min at 60-watt microwave power except for arginine residues, which were coupled twice each. Each deprotection reaction was carried out at 75° C. twice, once for 30 sec and then once for 3 min at 35-watt microwave power. Once synthesis was complete, the resin was washed with DMF (3×50 mL) and the N-terminus of the solid phase bound peptide was acetylated with acetic anhydride in the presence of DIPEA at room temperature. After acetylation of the N-terminus, the peptide resin was washed with DMF (3×20 mL) and DCM (3×20 mL). The peptide was cleaved from the solid support by treatment with a cleavage cocktail consisting of trifluoroacetic acid (TFA): H₂O: triisopropylsilane (TIPS) (95%: 2.5%: 2.5%, 10 mL) for 3 h at room temperature. Excess TFA was removed by sparging with nitrogen. The cleaved peptide was precipitated via the addition of ice-cold diethyl ether and centrifuged at 3000 rpm for 5 min. The crude peptide pellet was washed thrice with cold diethyl ether (3×40 mL) and purified by RP-HPLC using a Varian 940-LC HPLC System fitted with a 445-LC Scale-up module and 440-LC fraction collector. Peptides were purified by semi-preparative HPLC on an RP-C18 column (10×250 mm, Phenomenex Jupiter) using a linear gradient of CH₃CN in 0.1% TFA/H₂O with a flow rate of 15 mL min 1. Detection was performed at 220 nm and 260 nm. (See Table 4 for yields).

TABLE 4
Yields of peptides synthesized on larger scale for conjugation

| PepGen Number | Yield |
|---|---|
| DPEP 2.1 | 57% |
| DPEP 2.2 | 67% |
| DPEP 2.3 | 54% |
| DPEP 2.4 | 59% |
| DPEP 2.9 | 61% |
| DPEP 2.10 | 64% |
| DPEP 2.11 | 65% |

### b) Synthesis of PMO-Peptide Conjugates

A 25-mer PMO antisense sequence for mouse dystrophin exon-23 (GGCCAAACCTCGGCTTACCTGAAAT (SEQ ID NO:30)) was used. The peptide was conjugated to the 3′-end of the PMO through its C-terminal carboxyl group. This was achieved using 2.5 and 2-fold equivalents of PyBOP and HOAt in NMP respectively in the presence of 2.5 eq of DIPEA and a 2-fold excess of peptide over PMO dissolved in DMSO was used. In a few examples, HBTU (2 equivalents) were used in place of PyBOP for activation of the C-terminal carboxyl group of the peptide. In general, to a solution of peptide (10 μmol) in N-methylpyrrolidone (NMP, 100 μL) were added HBTU (76.6 μL of 0.3 M in NMP), HOAt in (66.7 μL of 0.3 M NMP), DIPEA (4.0 μL) and PMO (400 μL of 10 mM in DMSO). The mixture was left for 2 h at 40° C. and the reaction was quenched by the addition of 0.1% TFA (1 mL). The reaction was purified on a cation exchange chromatography column (Resource S 6 mL column, GE Healthcare) using 25 mM sodium phosphate buffer (pH 7.0) containing 25% acetonitrile. A sodium chloride solution (1 M) was used to elute the conjugate from the column at a flow rate of 6 mL min-1. The removal of excess salts from the peptide-PMO conjugate was afforded through the filtration of the fractions collected after ion exchange using an Amicon® ultra-15 3K centrifugal filter device. The conjugate was lyophilized and analyzed by MALDI-TOF. The conjugates were dissolved in sterile water and filtered through a 0.22 μm cellulose acetate membrane before use. The concentration of peptide-PMO was determined by the molar absorption of the conjugates at 265 nm in 0.1 N HCl solution. Overall yields (Table 5) were 29-46% based on PMO.

TABLE 5
Yields of P-PMO conjugates synthesized on larger scale for in vivo
analysis (The yields are based on dried weight of the lyophilised purified
ppmo. The purity for the PPMOs is greater than 95% as ascertained by
normal phase HPLC at 220 nm and 260 nm.

| Peptide Conjugate | Yield |
|---|---|
| DPEP 2.1-PMO | 34% |
| DPEP 2.2-PMO | 32% |
| DPEP 2.3-PMO | 39% |
| DPEP 2.4-PMO | 42% |
| DPEP 2.9-PMO | 46% |
| DPEP 2.10-PMO | 29% |
| DPEP 2.11-PMO | 30% |

### 1.7 In Vivo Assessment of Dystrophin Restoration by P-PMO

Experiments were conducted in the Biomedical Sciences Unit, University of Oxford, under Home Office Project Licence authorisation following institutional ethical review. Mice were housed in a minimal disease facility; the environment was temperature controlled with a 12 hour light-dark cycle. All animals received commercial rodent chow and water ad libitum.

Experiments were performed in 10-12 week old female mdx mice. Mdx mice were restrained prior to a single tail vein injection of 10 mg/kg of P-PMO. One week post injection mice were sacrificed and TA, heart and diaphragm muscles removed and snap frozen in dry-ice cooled isopentane and stored at −80° C.

### 1.8 Western Blot Analysis

To assess the duration of dystrophin restoration following a single administration, one-third of the muscle (for TA and diaphragm) or ninety 7 μm thick transverse cryosections (for heart) were lysed in 300 μl buffer (50 mM Tris pH 8, 150 mM NaCl, 1% NP40, 0.5% sodium deoxycholate, 10% SDS and protease/phosphatase inhibitors) prior to centrifuging at 13000 rpm (Heraeus, #3325B) for 10 min. Supernatant was collected and heated at 100° C. for 3 min. Protein was quantified by BCA method and 40 μg protein/sample were resolved in a NuPage a 3-8% Tris-Acetate gel as previously described (19). Proteins were transferred to a 0.45 μm pore size PVDF membrane for 1 h at 30V followed by 1 h at 100V, and probed with monoclonal anti-dystrophin (1:200, NCL-DYS1, Novocastra) and anti-vinculin (loading control, 1:100 000, hVIN-1, Sigma) antibodies as previously described (37). Secondary antibody IRDye 800CW goat anti-mouse was used at a dilution of 1:20000 (LiCOR).

The levels of dystrophin restoration in P-PMO treated mdx mice were expressed as relative to the levels of C57BL/10 wildtype control mice, considered as 100%. For this, a standard curve was generated by including 5 serial C57BL/10 protein dilutions in parallel to the P-PMO treated mdx samples. Dilution series were as follows: 75%, 40%, 15%, 5% or 0% respectively of the 40 μg total protein loaded per lane were from C57BL/10 protein lysates and the remaining from un-treated mdx protein lysates. These standards were aliquoted and used in each western blot in parallel to the treated mdx samples. For all standards and treated samples, Dystrophin intensity quantification was performed by Fluorescence Odyssey imaging system and normalized by calculating the ratio to the Vinculin fluorescence intensity in all samples. Standard normalized values were plotted against their known concentration of dystrophin to obtain the mathematical expression of best fit and this expression used to interpolate the normalized values of each sample of P-PMO treated mdx mice.

### 1.9 RT-qPCR Analysis of In Vivo Dmd Exon 23 Skipping

Quantification of exclusion of exon 23 from the mouse Dmd transcript was performed on skeletal muscle and heart tissue treated with peptide-PMO. Briefly, RNA was extracted from homogenised tissue using Trizol-based extraction method and cDNA synthesised using random primers. Primer/probes were synthesised by Integrated DNA Technologies and designed to amplify a region spanning exon 23-24 representing unskipped product (mDMD23-24, see Table 6), or to amplify specifically transcripts lacking exon 23 using a probe spanning the boundary of exon 22 and 24 (mDMD22-24). Levels of respective transcripts were determined by calibration to standard curves prepared using known transcript quantities, and skipping percentages derived by [skip]/[skip+unskip].

TABLE 6
Primer and probe sequences for quantification of exon 23
skipping by nested RT-PCR or quantitative RT-PCR methods.

| | | |
|---|---|---|
| | | Sequence |
| Assay ID | Primer Sequence (5'-3') | ID NO. |

Nested RT-PCR

| | | |
|---|---|---|
| Exon20Fo | CAGAATTCTGCCAATTGCTGAG | 31 |
| Exon26Ro | TTCTTCAGCTTGTGTCATCC | 32 |
| Exon20Fi | CCCAGTCTACCACCCTATCAGAGC | 33 |
| Exon26Ri | CCTGCCTTTAAGGCTTCCTT | 34 |

qRT-PCR

| | | |
|---|---|---|
| mDMD23- | Primer 1 CAGGCCATTCCTOTTTCAGG | 35 |
| 24 | Primer 2 GAAACTTTCCTCCCAGTTGGT | 36 |
| | Probe | 37 |
| | /5FAM/TCAACTTCA/ZEN/GCCATCCATTTCTGTAAGGT/3IABkFQ/ | |
| mDMD22- | Primer 1 CTGAATATGAAATAATGGAGGAGAGACTCG | 38 |
| 24 | Primer 2 CTTCAGCCATCCATTTCTGTAAGGT | 39 |
| | Probe /5FAM/ATGTGATTC/ZEN/TGTAATTTCC/3IABkFQ/ | 40 |

### 2. Results

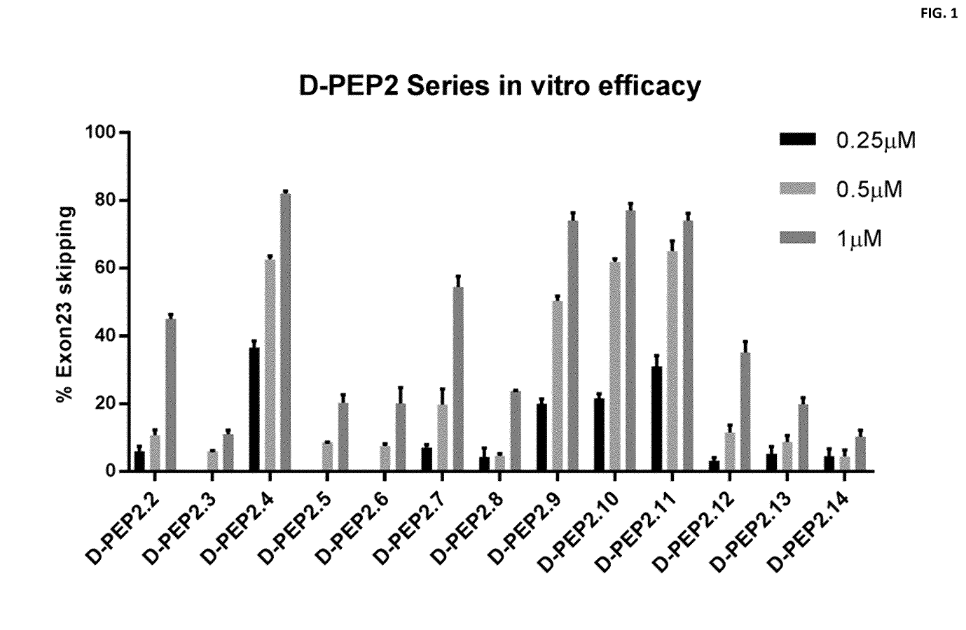

The results provided herein demonstrate a clear dose response effect of the peptide-PMO conjugates generated herein in exon skipping activity within cells (FIG. 1). This also highlights that all of the DPEP2 series of peptides, i.e. the peptides of the invention, have sufficient cell-penetrating efficacy in cells to be considered for therapeutic use.

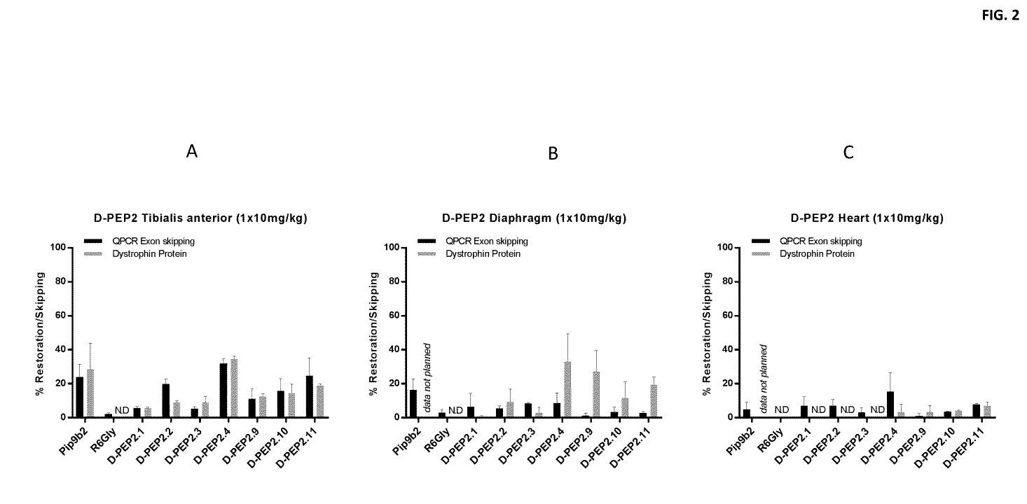

The results provided herein further highlight the activity of the peptide-PMO conjugates of the invention in vivo in a relevant mouse model of disease (FIG. 2). Overall the results suggest that activity of the peptide conjugates of the invention is greatest in tibialis anterior>diaphragm>heart. These results demonstrate that DPEP's of the invention have good exon skipping activity in vivo and provide an increase in dystrophin protein expression in vivo. Furthermore, the DPEPs of the invention compare favourably in at least skeletal muscle with previous cell-penetrating peptides such as ‘PIP’ peptides and R6Gly, when used in the same conjugate. It is notable that all of the peptide conjugates of the invention have higher activity than the known R6Gly comparator when used in the same conjugate.

Therefore the DPEP2 peptides of the invention provide promising cell-penetrating peptides for improving the efficacy of therapeutic conjugates for the treatment of neuromuscular disorders in humans.

1-25. (canceled)

26. A peptide having a total length of 40 amino acid residues or less, the peptide comprising:
one cationic domain, wherein the cationic domain comprises RBRXRBRXB (SEQ ID NO:1), RBRXRBRXBRXRB (SEQ ID NO:2), RBRXRBRXBR (SEQ ID NO:3), RBRRXRBRXBRXRB (SEQ ID NO:4), RBRXRBRBRXRB (SEQ ID NO:5), RBRXRBRBRBRB (SEQ ID NO:6), RBRBRBRBRBRB (SEQ ID NO:7), RBRXRBRBRXR (SEQ ID NO:8), RBRRBRBRBRRB (SEQ ID NO:9), RBRRBRBRXBRXRB (SEQ ID NO:10), RBRRBRBRBBRXRB (SEQ ID NO:11), RBRRBRBRBBRBRB (SEQ ID NO:12), or any combination thereof; and
two hydrophobic domains, wherein each hydrophobic domain independently comprises YQFLI (SEQ ID NO: 13), FQILY (SEQ ID NO:14), ILFQY (SEQ ID NO: 15), or any combination thereof;
wherein one of the hydrophobic domains is positioned at the C-terminus of the peptide and one of the hydrophobic domains is positioned at the N-terminus of the peptide, and wherein the two hydrophobic domains flank the cationic domain, wherein the N-terminus of the peptide is acetylated.

27. The peptide according to claim 26, wherein each hydrophobic domain has a length of 5 amino acids.

28. The peptide according to claim 26, wherein each hydrophobic domain independently consists of one of the following sequences: YQFLI (SEQ ID NO:13), FQILY (SEQ ID NO:14), ILFQY (SEQ ID NO:15), or any combination thereof.

29. The peptide according to claim 26, wherein the cationic domain has a length of between 9 to 14 amino acid residues.

30. The peptide according to claim 26, wherein the cationic domain consists of one of the following sequences: RBRXRBRXB (SEQ ID NO:1), RBRXRBRXBRXRB (SEQ ID NO:2), RBRXRBRXBR (SEQ ID NO: 3), RBRRXRBRXBRXRB (SEQ ID NO:4), RBRXRBRBRXRB (SEQ ID NO:5), RBRXRBRBRBRB (SEQ ID NO: 6), RBRBRBRBRBRB (SEQ ID NO:7), RBRXRBRBRXR (SEQ ID NO:8), RBRRBRBRBRRB (SEQ ID NO: 9), RBRRBRBRXBRXRB (SEQ ID NO:10), RBRRBRBRBBRXRB (SEQ ID NO:11), RBRRBRBRBBRBRB (SEQ ID NO:12), or any combination thereof.

31. The peptide according to claim 26, wherein the peptide consists of one cationic core domain comprising a sequence selected from: RBRXRBRXB (SEQ ID NO:1), RBRXRBRXBRXRB (SEQ ID NO:2), RBRXRBRXBR (SEQ ID NO:3), RBRRXRBRXBRXRB (SEQ ID NO:4), RBRXRBRBRXRB (SEQ ID NO:5), RBRXRBRBRBRB (SEQ ID NO:6), RBRBRBRBRBRB (SEQ ID NO:7), RBRXRBRBRXR (SEQ ID NO:8), RBRRBRBRBRRB (SEQ ID NO:9), RBRRBRBRXBRXRB (SEQ ID NO:10), RBRRBRBRBBRXRB (SEQ ID NO: 11), RBRRBRBRBBRBRB (SEQ ID NO:12), and the two hydrophobic arm domains each independently consist of a sequence selected from: YQFLI (SEQ ID NO:13), FQILY (SEQ ID NO:14), ILFQY (SEQ ID NO:15).

32. The peptide according to claim 26, where the peptide consists of one of the following sequences:
(SEQ ID NO: 16)
YQFLIRBRXRBRXBYQFLI,
(SEQ ID NO: 17)
YQFLIRBRXRBRXBRXRBYQFLI,
(SEQ ID NO: 18)
YQFLIRBRXRBRXBRYQFLI,
(SEQ ID NO: 19)
YQFLIRBRRXRBRXBRXRBYQFLI,
(SEQ ID NO: 20)
YQFLIRBRXRBRBRXRBYQFLI,
(SEQ ID NO: 21)
YQFLIRBRXRBRBRBRBYQFLI,
(SEQ ID NO: 22)
YQFLIRBRBRBRBRBRBYQFLI,
(SEQ ID NO: 23)
YQFLIRBRXRBRBRXRYQFLI,
(SEQ ID NO: 24)
YQFLIRBRRBRBRBRRBYQFLI,
(SEQ ID NO: 25)
YQFLIRBRRBRBRXBRXRBYQFLI,
(SEQ ID NO: 26)
YQFLIRBRRBRBRBBRXRBYQFLI,
(SEQ ID NO: 27)
YQFLIRBRRBRBRBBRBRBYQFLI,
(SEQ ID NO: 28)
FQILYRBRRBRBRBBRBRBFQILY, and
(SEQ ID NO: 29)
YQFLIRBRRXRBRXBRXRBFQILY.

33. The peptide according to claim 26, wherein the peptide consists of the amino acid sequence YQFLIRBRXRBRXBYQFLI (SEQ ID NO:16).

34. The peptide according to claim 26, wherein the peptide consists of the amino acid sequence YQFLIRBRXRBRXBRXRBYQFLI (SEQ ID NO:17).

35. The peptide according to claim 26, wherein the peptide consists of the amino acid sequence YQFLIRBRXRBRXBRYQFLI (SEQ ID NO:18).

36. The peptide according to claim 26, wherein the peptide consists of the amino acid sequence YQFLIRBRRXRBRXBRXRBYQFLI (SEQ ID NO:19).

37. The peptide according to claim 26, wherein the peptide consists of the amino acid sequence YQFLIRBRXRBRBRXRBYQFLI (SEQ ID NO:20).

38. The peptide according to claim 26, wherein the peptide consists of the amino acid sequence YQFLIRBRXRBRBRBRBYQFLI (SEQ ID NO:21).

39. The peptide according to claim 26, wherein the peptide consists of the amino acid sequence YQFLIRBRBRBRBRBRBYQFLI (SEQ ID NO:22).

40. The peptide according to claim 26, wherein the peptide consists of the amino acid sequence YQFLIRBRXRBRBRXRYQFLI (SEQ ID NO:23).

41. The peptide according to claim 26, wherein the peptide consists of the amino acid sequence YQFLIRBRRBRBRBRRBYQFLI (SEQ ID NO:24).

42. The peptide according to claim 26, wherein the peptide consists of the amino acid sequence YQFLIRBRRBRBRXBRXRBYQFLI (SEQ ID NO:25).

43. The peptide according to claim 26, wherein the peptide consists of the amino acid sequence YQFLIRBRRBRBRBBRXRBYQFLI (SEQ ID NO:26).

44. The peptide according to claim 26, wherein the peptide consists of the amino acid sequence YQFLIRBRRBRBRBBRBRBYQFLI (SEQ ID NO:27).

45. The peptide according to claim 26, wherein the peptide consists of the amino acid sequence FQILYRBRRBRBRBBRBRBFQILY (SEQ ID NO:28).

46. The peptide according to claim 26, wherein the peptide consists of the amino acid sequence YQFLIRBRRXRBRXBRXRBFQILY (SEQ ID NO:29).

47. A conjugate comprising the peptide of claim 26 covalently linked to a therapeutic molecule.

48. The conjugate according to claim 47, further comprising a linker covalently linking the peptide to the therapeutic molecule.

49. The conjugate according to claim 47, wherein the linker is selected from G, BC, XC, C, GGC, BBC, BXC, XBC, X, XX, B, BB, BX and XB.

50. The conjugate according to claim 48, wherein the therapeutic molecule is selected from: a nucleic acid, peptide nucleic acid, antisense oligonucleotide, short interfering RNA, micro RNA, peptide, cyclic peptide, protein, pharmaceutical or drug.

51. The conjugate according to claim 47, wherein the therapeutic molecule is an antisense oligonucleotide comprising the sequence GGCCAAACCTCGGCTTACCTGAAAT (SEQ ID NO:30).

52. The conjugate according to claim 48, wherein the therapeutic molecule is an antisense oligonucleotide comprising the sequence GGCCAAACCTCGGCTTACCTGAAAT (SEQ ID NO:30).

53. The conjugate according to claim 47, wherein the therapeutic molecule is a PMO.

54. A method for the treatment of DMD, the method comprising administering the conjugate of claim 47 to a subject in need thereof.

55. An isolated nucleic acid encoding a cell-penetrating peptide consisting of the amino acid sequence
(SEQ ID NO: 16)
YQFLIRBRXRBRXBYQFLI,
(SEQ ID NO: 17)
YQFLIRBRXRBRXBRXRBYQFLI,
(SEQ ID NO: 18)
YQFLIRBRXRBRXBRYQFLI,
(SEQ ID NO: 19)
YQFLIRBRRXRBRXBRXRBYQFLI,
(SEQ ID NO: 20)
YQFLIRBRXRBRBRXRBYQFLI,
(SEQ ID NO: 21)
YQFLIRBRXRBRBRBRBYQFLI,
(SEQ ID NO: 22)
YQFLIRBRBRBRBRBRBYQFLI,
(SEQ ID NO: 23)
YQFLIRBRXRBRBRXRYQFLI,
(SEQ ID NO: 24)
YQFLIRBRRBRBRBRRBYQFLI,
(SEQ ID NO: 25)
YQFLIRBRRBRBRXBRXRBYQFLI,
(SEQ ID NO: 26)
YQFLIRBRRBRBRBBRXRBYQFLI,
(SEQ ID NO: 27)
YQFLIRBRRBRBRBBRBRBYQFLI,
(SEQ ID NO: 28)
FQILYRBRRBRBRBBRBRBFQILY,
(SEQ ID NO: 29)
YQFLIRBRRXRBRXBRXRBFQILY,
wherein the N-terminus of the peptide is acetylated.

56. An expression vector comprising the isolated nucleic acid of claim 55.

57. A host cell comprising the expression vector of claim 56.
